(19) United States
(12) Patent Application Publication
(10) Pub. No.: US 2008/0027276 A1
(43) Pub. Date: Jan. 31, 2008

(54) ENDOSCOPIC PROBE INTEGRATING A COMPACT OBJECTIVE

(75) Inventor: Jean ROVEGNO, LA CIOTAT (FR)

Correspondence Address: AKIN GUMP STRAUSS HAUER & FELD L.L.P., ONE COMMERCE SQUARE, 2005 MARKET STREET, SUITE 2200, PHILADELPHIA, PA, 19103, US

(73) Assignee: TOKENDO, LA CIOTAT (FR)

(21) Appl. No.: 11/782,662

(22) Filed: Jul. 25, 2007

(30) Foreign Application Priority Data
Jul. 27, 2006 (FR) 06 06878

(51) Int. Cl. A61B 1/04

(52) U.S. Cl. 600/109

(57) ABSTRACT

An endoscopic probe is provided including a distal end (11, 31) having an image sensor (1) with a substantially rectangular light-sensitive surface (2) associated with an objective arranged for transmitting a light beam having a proximal section substantially identical to the light-sensitive surface of the image sensor. The objective includes at least one distal lens (32, 33) produced from a circular lens, wherein parts of the circular lens that are not passed through by light rays transmitted by the objective to the light-sensitive surface (2) have been removed, so as to reduce the lateral dimensions of a distal part of the objective, in order to particularly house a high-power lighting device.

### BACKGROUND OF THE INVENTION

The present invention relates to a videoendoscopic probe integrating an objective housed either in the distal terminal end of the probe, or in a head that is removable from and lockable onto the distal terminal end.

The present invention applies particularly, but not exclusively, to industrial endoscopy.

In the following description, the words “endoscope” or “fiberscope” mean a rigid or flexible probe, intended to be introduced into a dark cavity and enabling its user to observe through an eyepiece the image of a target located in the cavity. Such a probe therefore integrates a lighting device for lighting up the target and an optical device, supplying the user with an image of the target. The optical device comprises a distal objective, a proximal eyepiece, and an image transporting device for transporting images between the objective and the eyepiece. The image transporting device is either rigid, made up of a series of lenses, or flexible, made up of a bundle of aligned optical fibers. The lighting device generally comprises a bundle of lighting fibers comprising a distal end suitably oriented proximate to the distal objective to illuminate the target, and a proximal end capable of being connected to a white light generator.

In the following description, the word “videoendoscope” means a flexible or rigid probe enabling its user to observe on a video screen the image of a target located in a dark cavity.

For this purpose, a videoendoscope comprises a lighting device for lighting up the target identical to that of an endoscope or of a fiberscope and an optoelectronic device supplying the user with a video image of the target. A videoendoscope can be made up either of an endoscope or of a fiberscope the eyepiece of which is connected onto the objective of an endoscopy video camera, or have a specific structure generally comprising:

a distal terminal end,

a generally flexible inspection tube, the distal end of which is fitted into the distal terminal end,

a control handle fitted into the proximal end of the inspection tube,

a flexible connection umbilical tube whose distal end is fitted into the control handle,

a control panel, and

a video monitor.

The distal terminal end houses an optoelectronic device comprising particularly an image sensor associated with an objective forming an image on the light-sensitive surface of the sensor. The umbilical tube has a proximal end intended to be connected to an external case integrating particularly a light generator and a power supply source. The light generated by the light generator is transmitted towards the distal terminal end by a bundle of lighting fibers housed in the umbilical tube, in the control handle, and in the inspection tube.

A video processor is linked to the distal image sensor by a multiconductor electric lead, and transforms the electric signal supplied by the image sensor into a useful video signal. The synchronization of the video processor with the image sensor is adjusted according to the length of the connecting lead between the latter.

The control handle preferably integrates the video monitor and the control panel enabling the operation of the video processor to be adjusted particularly according to the color temperature of the lighting of the target by the distal end of the bundle of lighting fibers.

The videoendoscopic probes supplying a color video image can have two different types of architecture. According to the first type of architecture, the videoendoscopic probes are equipped with an image sensor of “monochrome” CCD type supplying an electric signal containing only a piece of luminance information. Obtaining a useful video signal exploitable on a color monitor then involves using a specific light generator sequentially supplying flashes corresponding to the three primary colors, a device for capturing the electric signals supplied sequentially by the image sensor during the successive flashes, and a video processor arranged for continuously storing the last three monochrome video frames and reconstituting from these three frames a useful video signal directly exploitable by a color video monitor.

The videoendoscopic probes having the second type of architecture are the most common ones. They are equipped with an image sensor of “interline transfer tricolor” CCD type which is associated with a white light generator. The image sensor supplies an electric signal containing luminance and chrominance information which is processed by the video processor to supply a useful video signal directly exploitable by a color video monitor.

Whatever their type of architecture, the videoendoscopic probes may further comprise some or all of the following elements.

Thus, the distal terminal end of the probe can be associated with an articulated distal tip deflection enabling the orientation of the distal terminal end to be modified. The control handle then comprises mechanical or electromechanical control means enabling the tip deflection to be activated.

The distal terminal end can also be designed to receive removable optical heads integrating an objective, these heads being adaptable to its distal end. Changing the optical head particularly enables the optical field covered and/or the directions of the optical viewing and lighting axes of the videoendoscopic probe to be modified. Mechanical devices position and lock the heads onto the distal end of the distal terminal end.

In particular, these mechanical devices ensure the continuity of the optical pathways and of the lighting pathways between the videoendoscopic probe and the removable head. They are further arranged to prevent any possibility of the removable head accidentally unlocking, and not cause any pollution of the image sensor by stray light rays coming from the light pathway.

The videoendoscopic probes can also comprise a digital device for freezing, recording and processing images; this device can be either a simple laptop computer equipped with a video input, or a dedicated system, managed by the control panel implanted onto the control handle of the videoendoscopic probe.

A metrology device can also be associated with the videoendoscopic probe to directly measure the actual dimensions of an element of a target being inspected on a previously frozen video image of the target. The metrology device comprises an optical means integrated into the distal terminal end of the videoendoscopic probe, or preferably, into a specific removable head adaptable to the distal end of the distal terminal end. The optical means enables either the image of the target viewed on the video monitor to be split by a stereo vision method, or an auxiliary image characterizing the actual position of the target in the optical field covered by the probe to be inserted into the image. A specific calculation program managed by the image processing device enables the user to point on the video image either the ends of the two images of the target generated by the optical image splitting means, or the ends of the video image of the target and of the auxiliary image. The program then implements a calculation algorithm enabling the actual dimensions of the target to be deduced from the pointing.

Historically speaking, the lighting devices used in rigid endoscopy in the 1950s consisted of a filament lamp integrated into the distal terminal end of a lateral-viewing endoscope. The lamp was generally associated with a mirror laterally deviating its luminous flux so that the lighting field covers the optical field of the endoscope. Such devices are particularly described, for example, in U.S. Pat. Nos. 2,779,327 and 3,096,756. Certain devices still used today are based on this principle and implement compact halogen-type lamps.

As flexible optical fibers are now widely used, the use of lighting devices comprising a bundle of lighting fibers has become widespread, both in rigid endoscopy and in fiberoptic endoscopy or in videoendoscopy. The bundle of lighting fibers is housed in the endoscopic probe, the distal end of the bundle being integrated into the distal terminal end of the probe. Thus, the distal end of the bundle of lighting fibers illuminates the target when the proximal end of the bundle is connected to a light generator.

The miniaturization of lighting diodes of LED type (Light Emitting Diode) has revived use of the first lamp-based lighting devices used in endoscopy. This brings real progress in terms of portability and power consumption, such progress resulting from the removal of the halogen or xenon lamp-based light generator associated with a bundle of optical fibers.

U.S. Pat. Nos. 6,260,994 and 6,318,887 describe a first type of LED-based lighting device architecture suited to the videoendoscopic probes. The lighting devices described in these documents comprise a panel of LEDs disposed on a concave medium, and an optical device focusing the luminous fluxes emitted by the diodes on the proximal end of a bundle of lighting optical fibers.

U.S. Patent Application Publication US2002/0120181 and U.S. Pat. No. 6,730,019 describe a second type of architecture. In this type of architecture, several LEDs are integrated longitudinally into the handle of an endoscope. Each diode is associated with an objective lens and a semi-reflecting prism, arranged so as to channel the luminous fluxes emitted by the diodes onto the proximal end of a bundle of lighting optical fibers housed in the inspection tube of the endoscope.

In a third type of architecture, the bundle of lighting optical fibers is removed by directly disposing one or more LEDs in the distal terminal end of an endoscopic probe. Thus, U.S. Pat. No. 5,908,294 describes a rigid lateral-viewing videoendoscopic probe whose distal terminal end houses two LEDs, the lighting field of which covers the optical field of the probe. U.S. Pat. Nos. 5,264,925; 6,033,087 and 6,449,006, and U.S. Patent Application Publications US2003/0035048 and US2004/0064018 describe axial-viewing videoendoscopic probes comprising a lighting device comprising several LEDs disposed in a concentric manner around the objective of the probe.

In a fourth type of architecture described in International Patent Application Publications WO2005/032356, WO2006/001377 and WO2006/046559, an objective made up of traditional circular lenses, and a lighting device made up of several LEDs in SMC (surface-mounted component) boxes, are integrated into a removable distal head.

Most of these lighting devices can be used in the videoendoscopic probes equipped with a monochrome CCD sensor and with a sequential tricolor lighting device. In this case, three LEDs (or three groups of LEDs) are provided to send light respectively in spectral bands characterizing three complementary colors such as red/green/blue.

It is also known to dispose LEDs all emitting in white light, in a ring around an objective associated with a CCD sensor in traditional color video cameras, and in videoendoscopic probes equipped with a monochrome CCD sensor.

However, the space available around an objective made up of traditional lenses in a videoendoscopic probe distal terminal end or in a removable distal head lockable onto such a distal terminal end, does not enable LEDs sufficiently voluminous to supply satisfactory candle power to be implemented, even if the LEDs are packed in SMC boxes. Thus, a removable distal head with a diameter of 6 mm enables only 6 to 7 LEDs packed in SMC boxes of 1.6×0.8 mm to be housed. Such a set of diodes supplies a global candle power in the order of 2 candelas. Such a power proves to be insufficient in excess of a viewing distance in the order of 10 to 20 mm. Beyond this distance, the user is forced to artificially increase the sensitivity of the probe by implementing for example an electronic device for integrating successive video images that, in excess of a certain integration time, creates totally unacceptable visual fatigue.

### BRIEF SUMMARY OF THE INVENTION

The present invention aims to integrate into the distal end of a videoendoscopic probe a device, such as a lighting device, comprising one or two high-power light-emitting diodes.

Thus, one principle of the present invention is to reduce the dimensions of the distal circular lenses of the objective of the videoendoscopic probe, so as to free up a sufficient volume to particularly house high-power lighting diodes.

More precisely, the present invention provides an endoscopic probe comprising a distal end comprising an image sensor having a substantially rectangular light-sensitive surface associated with an objective arranged for transmitting a light beam having a proximal section substantially identical to the light-sensitive surface of the image sensor.

According to one embodiment of the present invention, the objective comprises at least one first distal lens produced from a circular lens, parts of which that are not passed through by light rays transmitted by the objective to the light-sensitive surface have been removed, so as to reduce the lateral dimensions of a distal part of the objective.

According to one embodiment of the present invention, the first distal lens of the objective is produced from a circular lens having undergone two symmetrical lateral milling operations, the distance between them being substantially equal to the width of the widest section of the part of the light beam passing through the distal part of the objective and covering the light-sensitive surface.

According to one embodiment of the present invention, the objective comprises a second distal lens having a section identical to that of the first distal lens.

According to one embodiment of the present invention, the objective comprises at least one proximal lens of circular section the diameter of which is substantially identical to a diagonal of the light-sensitive surface.

According to one embodiment of the present invention, the objective comprises at least one proximal lens machined from a circular lens so as to have a width substantially identical to that of the light-sensitive surface.

According to one embodiment of the present invention, the objective comprises a first diverging distal lens, a second converging distal lens, and a converging proximal lens, the distal lenses being produced from circular lenses each having undergone two symmetrical lateral milling operations, the distance between them being substantially equal to the width of the widest section of a distal part of the light beam passing through the objective and covering the light-sensitive surface.

According to one embodiment of the present invention, the endoscopic probe comprises a lighting device comprising one or two high-power light-emitting diodes housed at least partly in the volume of the parts removed from the distal lens.

According to one embodiment of the present invention, the lighting device comprises for each light-emitting diode a field lens, a printed circuit supporting the diode and a distal port protecting the diode.

According to one embodiment of the present invention, the image sensor is housed in the distal end of the endoscopic probe.

According to one embodiment of the present invention, at least one distal part of the objective and the lighting device are disposed in a head arranged for being fixed in a removable manner onto a distal terminal end of the endoscopic probe.

According to one embodiment of the present invention, the removable head comprises electrical connection means arranged for cooperating with corresponding electrical connection means provided in the distal terminal end to ensure the electrical connection of the lighting device when the removable head is fixed onto the distal terminal end.

According to one embodiment of the present invention, the endoscopic probe comprises a source of power in pulsed current to deactivate the lighting device when the image sensor is not in an acquisition phase.

The present invention also relates to a removable head lockable onto an endoscopic probe distal terminal end, comprising at least one distal part of an objective arranged for transmitting a light beam covering a substantially rectangular light-sensitive surface of an image sensor.

According to one embodiment of the present invention, the objective comprises at least one first distal lens produced from a circular lens, parts of which that are not passed through by light rays transmitted by the objective to the light-sensitive surface have been removed, so as to reduce the lateral dimensions of the distal part of the objective.

According to one embodiment of the present invention, the first distal lens of the objective is produced from a circular lens having undergone two symmetrical lateral milling operations, the distance between them being substantially equal to the width of the widest section of the part of the light beam passing through the distal part of the objective and covering the light-sensitive surface.

According to one embodiment of the present invention, the objective comprises a second distal lens having a section identical to that of the first distal lens.

According to one embodiment of the present invention, the objective comprises at least one proximal lens of circular section, the diameter of which is substantially identical to a diagonal of the light-sensitive surface.

According to one embodiment of the present invention, the objective comprises at least one proximal lens machined from a circular lens, so as to have a width substantially identical to that of the light-sensitive surface.

According to one embodiment of the present invention, the objective comprises a first diverging distal lens, a second converging distal lens, and a converging proximal lens, the distal lenses being produced from circular lenses each having undergone two symmetrical lateral milling operations, the distance between them being substantially equal to the width of the widest section of a distal part of the light beam passing through the objective and covering the light-sensitive surface.

According to one embodiment of the present invention, the removable head comprises a lighting device comprising one or two high-power light-emitting diodes housed at least partly in the volume of the parts removed from the distal lens.

According to one embodiment of the present invention, the lighting device comprises for each light-emitting diode a field lens, a printed circuit supporting the diode and a distal port protecting the diode.

According to one embodiment of the present invention, the removable head comprises electrical connection means arranged for cooperating with corresponding electrical connection means provided in the distal terminal end of the probe to ensure the electrical connection of the lighting device when the removable head is fixed onto the distal terminal end.

### BRIEF DESCRIPTION OF THE SEVERAL VIEWS OF THE DRAWINGS

The foregoing summary, as well as the following detailed description of the invention, will be better understood when read in conjunction with the appended drawings. For the purpose of illustrating the invention, there are shown in the drawings embodiments which are presently preferred. It should be understood, however, that the invention is not limited to the precise arrangements and instrumentalities shown. In the drawings:

FIG. 1 is a longitudinal cross-sectional view of the distal end of a videoendoscopic probe comprising a distal terminal end associated with a classic removable head,

FIG. 2 is a front view of the distal end of the distal terminal end represented in FIG. 1,

FIG. 3 is a front view of the distal end of the removable head represented in FIG. 1,

Fig. is a longitudinal cross-sectional view of the distal end of a videoendoscopic probe comprising a distal terminal end associated with a removable head according to the present invention,

FIG. 5 is a front view of the distal end of the removable head represented in FIG. 4,

FIG. 6 is a perspective view of the distal end of the distal terminal end represented in FIG. 1,

FIGS. 7 and 8 are longitudinal cross-sections of a removable head according to the present invention, lockable onto the distal end of a probe represented in FIG. 6,

FIGS. 9 and 10 are perspective views of the removable distal head represented in FIGS. 7 and 8,

FIGS. 11A, 11B, 11C are timing diagrams of control signals applied to the image sensor of the videoendoscopic probe,

FIG. 12 is a schematic wiring diagram of a pulsed power supply circuit of the lighting device.

### DETAILED DESCRIPTION OF THE INVENTION

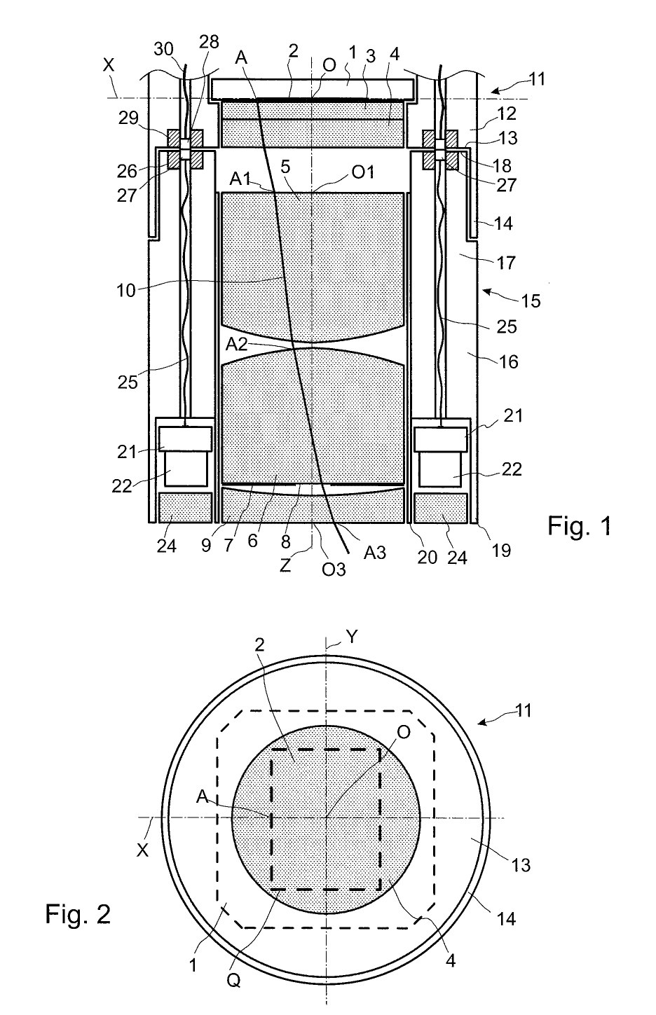

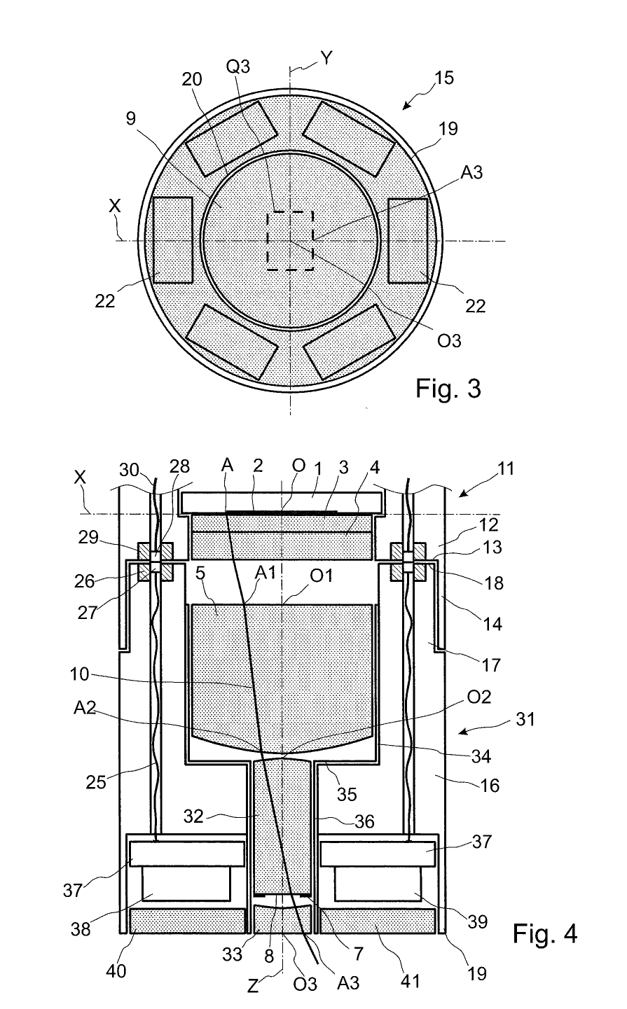

FIGS. 1 to 3 represent the distal end of a classic videoendoscopic probe. In FIGS. 1 to 3, the distal end of the videoendoscopic probe comprises a distal terminal end 11 associated with a removable distal head 15 lockable onto the distal terminal end. In FIGS. 1 and 3, the removable distal head 15 classically comprises a lighting device comprising several low-power light-emitting diodes (LED) 22 (six in the example in FIG. 3) packed in compact SMC boxes spread in a ring around the objective.

In FIGS. 1 and 2, the distal terminal end 11 comprises a tubular cylindrical body 12 having a distal transverse partition 13. The body 12 houses an image sensor 1 comprising a rectangular light-sensitive surface 2 protected by a transparent plate 3, and a circular infrared filter 4 having a diameter at least equal to the diagonal of the light-sensitive surface 2 of the image sensor. The infrared filter 4 is fixed onto the partition 13 in front of a central circular orifice formed in the latter. The distal terminal end 11 of the probe is extended by a cylindrical tube 14 used as a receptacle for a proximal part 17 of the removable head 15.

In FIGS. 1 and 3, the removable head 15 comprises a tubular cylindrical body 16 having an external diameter substantially identical to the distal terminal end 11. The body 16 houses a cylindrical mount 20 of an objective comprising, successively centered along an optical axis OZ, a converging proximal lens 5, a converging median lens 6, an aperture diaphragm 8 installed in an opaque disc 7 and a diverging distal lens 9. All the optical components of the objective have a circular section having a diameter substantially identical to that of the infrared filter 4.

The proximal part 17 of the removable head 15 has a slightly smaller diameter than the interior diameter of the distal tube 14, so as to be capable of being introduced into it until a proximal face 18 of the removable head 15 comes in contact with the distal partition 13 of the distal terminal end 11.

The distal end of the tubular body 16 is extended by a cylindrical tube 19 delimiting a distal volume located between the internal face of the tube 19 and the external face of the cylindrical mount 20 of the objective. This distal volume enables a lighting device to be housed comprising a printed circuit 21 in the form of a ring supporting six LEDs 22 packed in SMC boxes and protected by a distal transparent plate 24 in the shape of a ring. The printed circuit 21 is linked by two electrical conductors 25 to two contact pads 27 housed in insulating bases 26 arranged on the proximal face 18 of the removable head 15. When the removable head 15 is locked onto the distal terminal end 11, the two contact pads 27 of the removable head 15 come into electrical contact with two similar contact pads 28, housed in insulating bases 29 arranged on the distal partition 13 of the distal terminal end 11. The contact pads 28 are linked to an electrical power supply device by two electrical conductors 30.

The “useful” light rays, i.e. contributing to form the video image supplied by the videoendoscopic probe, are spread in an output window Q corresponding to the light-sensitive surface 2 of the image sensor 1. The window Q has a rectangular shape the width/height ratio of which is equal to 4/3. A light ray 10 ending at a point A on the edge of the window Q and contained in a longitudinal plane OXZ including an optical axis OZ of the optical system, successively passes through an input point A3 located on the distal face of the distal lens 9, through a point A2 located on the proximal face of the median lens 6, and through a point A1 located on the proximal face of the proximal lens 5.

The “useful” light rays successively pass through a series of homothetic rectangular windows of the window Q. The width/height ratio of these windows therefore remains equal to 4/3. The output window Q is centered on a point O located at the intersection of the optical axis OZ and of the light-sensitive surface 2 of the image sensor 1. The height of the window Q (width of the window Q along the axis OX) is equal to twice the length of the segment O-A. Before arriving in the window Q, the light rays pass through a window Q1, centered on a point O1 located at the intersection of the optical axis OZ and of the proximal face of the lens 5. The height of the window Q1 is therefore substantially equal to twice the length of the segment O1-A1. Before arriving in the window Q1, the light rays pass through a window Q2, centered on a point O2 located at the intersection of the optical axis OZ and of the proximal face of the lens 6. The height of the window Q2 is therefore substantially equal to twice the length of the segment O2-A2. The light rays enter the removable distal head 15 through an input window Q3 centered on a point O3 located at the intersection of the optical axis OZ and of the distal face of the lens 9. The height of the window Q3 is therefore substantially equal to twice the length of the segment O3-A3.

The principle of the present invention consists in reducing the dimensions of the distal part of the objective by removing non-useful parts of the circular lenses 6, 9, i.e. parts not passed through by the (useful) light rays transmitted to the light-sensitive surface of the sensor 1. The respective sections of the lenses 5, 6 and 9 can thus be reduced substantially to the rectangular windows Q1, Q2, Q3, without affecting the optical characteristics of the objective.

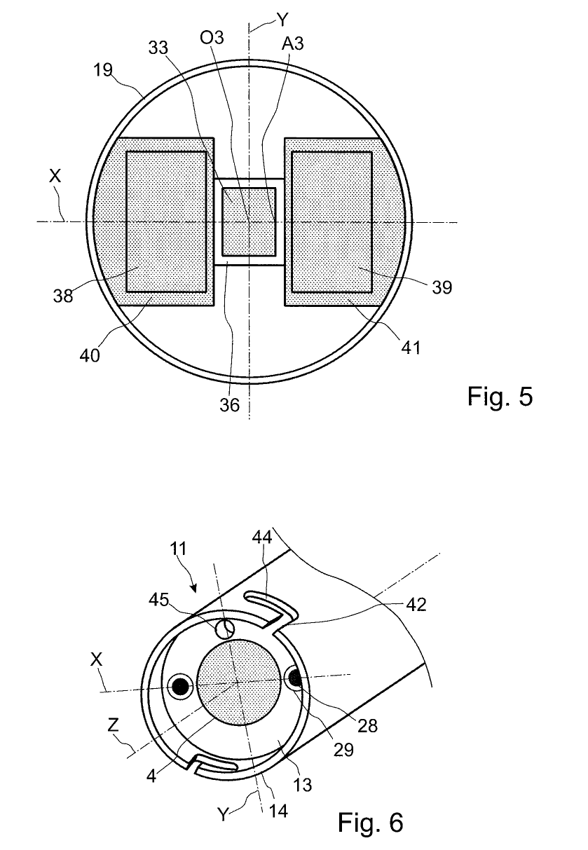

FIGS. 4 and 5 represent a videoendoscopic probe distal end according to one embodiment of the present invention. In FIGS. 4 and 5, the videoendoscopic probe distal end comprises a distal terminal end 11, associated with a removable distal head 31 lockable onto the distal terminal end 11. The distal terminal end 11 represented in FIG. 4 is identical in every respect to the distal terminal end previously described with reference to FIGS. 1 and 2.

The removable distal head 31 represented in FIGS. 4 and 5 differs from the removable head 15 described above with reference to FIGS. 1 and 3, in the following respects.

Only the proximal lens 5 keeps its circular shape, while the lenses 6 and 9 (FIGS. 1 and 2) have been respectively replaced by a median converging lens 32 and a diverging distal lens 33. The lenses 32, 33 each have a rectangular section substantially equal to the largest of the two windows Q2 and Q3. In the case of the optical system represented in FIGS. 1 and 3, the window Q3 is the largest.

Therefore, in the example in FIGS. 3 and 4, the lenses 32 and 33 have a section of dimensions substantially identical to those of the rectangular window Q3 centered on the point 03 located at the intersection of the optical axis OZ and of the distal face of the distal lens 33. As mentioned above, the window Q3 has a height equal to twice the length of the segment O3-A3 and a width/height ratio equal to 4/3.

The lenses 5, 32, 33 are fixed into a tubular objective mount having a circular section proximal part 34 housing the proximal lens 5 and a rectangular section distal part 36 housing the median lens 32 and the distal lens 33. The distal part 36 of the objective mount thus has walls parallel to a first longitudinal plane OXZ and to a second longitudinal plane OYZ perpendicular to the first longitudinal plane.

The distal volume between the internal face of the external tube 19 of the removable head and the walls of the distal part 36 of the objective mount, proves to be sufficiently large to be capable of housing a lighting device comprising two high-power LEDs 38, 39 each associated with a field lens in an SMC box, a printed circuit 37 supporting the two LEDs and distal ports 40, 41 protecting the LEDs. The SMC boxes in which the two diodes 38, 39 are packed have a surface which can reach, with identical probe diameters, 2.5 times that of the SMC boxes in which the six diodes 22 integrated into the removable head 15 are packed.

The present invention thus enables for example one or two LEDs packed in SMC boxes of 2.0×1.6 mm, each supplying a candle power of 9 candelas, to be housed in an axial-viewing removable distal head with a diameter of 6 mm.

In practice, the lenses 32 and 33 are manufactured from circular lenses, such as the lenses 6, 9, having a diameter substantially equal to the diagonal of the widest rectangular section of the part of the useful light beam passing through the two lenses. The lenses 6, 9 are machined by causing each of them to undergo two symmetrical lateral milling operations so as to form two opposite edges that are straight and symmetrical in relation to the center of the lens. These straight edges are spaced by a distance corresponding substantially to the width of the widest section of the useful light beam in the zone of the two lenses 6, 9, i.e. in the example of the Figs. the width of the window Q3. The edges of the lenses between the straight edges can remain circular.

FIGS. 6 to 10 show the principle of assembling and locking the distal head 31 onto the distal terminal end 11.

FIG. 6 represents the distal part of the distal terminal end 11, and in particular the external cylindrical tube 14 and the partition 13. In addition to the elements described with reference to FIGS. 1 and 2, the partition 13 comprises a blind cylindrical orifice 45 located in the longitudinal plane of symmetry OYZ.

In addition to the elements described with reference to FIGS. 1 and 2, the cylindrical tube 14 comprises two diametrically opposite slits in bayonet shape. Each slit comprises a longitudinal part 42 with an open distal end contained in a longitudinal plane of symmetry bisecting the two longitudinal planes of symmetry OXZ and OYZ, and a transverse part 44 comprising a closed end located in the longitudinal plane of symmetry OYZ.

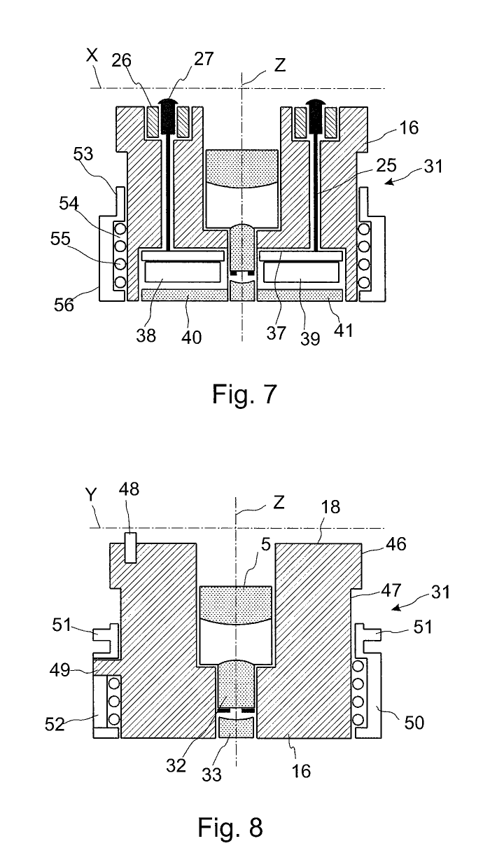

FIGS. 7 and 8 represent the axial-viewing removable head 31 which is lockable onto the distal terminal end 11 represented in FIG. 6. The removable head 31 comprises a central core 16 and an annular locking device 50.

FIG. 7 is a cross-section of the removable head 31 in the longitudinal plane of symmetry OXY, while FIG. 8 is a cross-section of the head according to the longitudinal plane of symmetry OXZ.

The central core 16 of the removable head 31 comprises an axial orifice housing the objective mount as described previously with reference to FIGS. 4 and 5.

In addition to the elements described with reference to FIGS. 4 and 5, the proximal face 18 of the central core 16 comprises a pilot point 48 located in a longitudinal plane of symmetry OYZ.

The central core 16 comprises a distal part 47 comprising a hollow located around the distal part 36 of the objective mount. This hollow houses the lighting device as described previously with reference to FIGS. 4 and 5.

The central core 16 comprises a proximal part 46 arranged for being engaged into the distal tube 14 of the distal terminal end 11. The proximal part 46 has a diameter greater than that of the distal part 47 of the core. The distal part 47 comprises an external radial finger 49 intended to longitudinally guide the annular locking device 50.

The locking device 50 which surrounds the distal part 47 of the central core 16 has a proximal cylindrical part 53 having an external diameter substantially identical to the external diameter of the proximal part 46 of the central core 16, and a distal cylindrical part 56 having an external diameter substantially identical to that of the distal terminal end 11. The proximal part 53 of the locking device 50 comprises two diametrically opposite external radial fingers 51 located in the longitudinal plane of symmetry OYZ. The fingers 51 are arranged to engage into and simultaneously circulate in the two bayonet-shaped slits 42, 44 made in the distal tube 14 of the distal terminal end 11. The distal part 56 of the locking device 50 comprises a closed longitudinal slit 52 in which the radial finger 49 fitted into the distal part 47 of the central core 16 circulates. The distal part 56 also comprises an internal annular housing 54 containing a coil spring 56 pressing on the radial finger 49 so as to exert a longitudinal pressure tending to take the finger back towards the proximal end of the longitudinal slit 52.

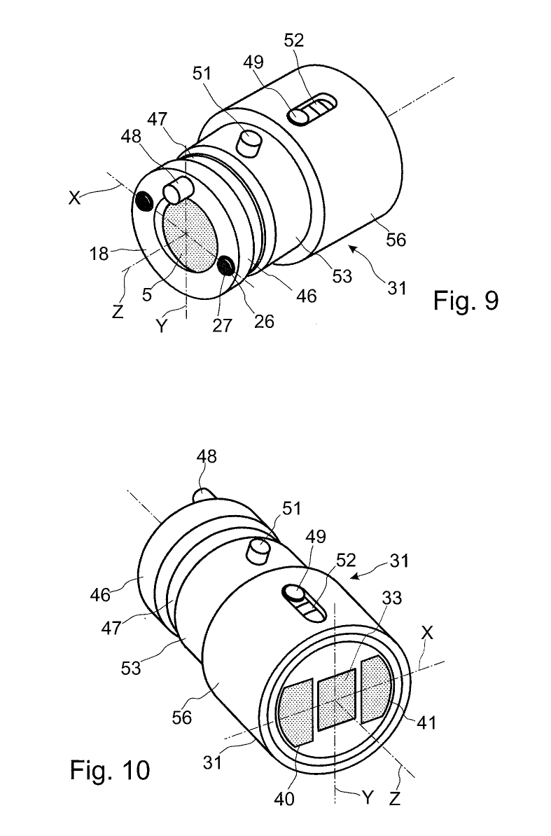

FIGS. 9 and 10 show the operation of locking the removable head 31 represented on FIGS. 7 and 8, onto the distal terminal end 11 represented in FIG. 6.

During an insertion phase, the proximal cylindrical part 46 of the central core 16 of the removable head 31 is inserted into the distal end of the tube 14 of the distal terminal end 11 until the two radial fingers 51 fitted into the proximal part 53 of the locking device 50 are engaged in the distal ends of the open longitudinal slits 42 made in the distal tube 14.

During a next compression phase, the proximal part 46 of the central core 16 of the removable head 31 is pushed as far as possible into the distal part of the tube 14 of the distal terminal end 11 by a longitudinal pressure exerted on the distal part 56 of the locking device 50. At the end of the compression phase, the respective positions of the various elements of the distal terminal end 11 and of the removable head 31 are then in the following configuration.

The coil spring 55 housed in the distal part 56 of the annular locking device 50 is compressed to a maximum. The radial finger 49 fitted into the distal part 47 of the central core 16 presses on the distal end of the closed longitudinal slit 52 made in the distal part 56 of the annular locking device 50. The two radial fingers 51 fitted into the proximal part 53 of the annular locking device 50 press on the proximal ends 43 of the longitudinal slits 42 made in the distal tube 14 of the distal terminal end 11. The proximal face of the proximal part 53 of the annular locking device 50 presses on the distal face of the proximal part 46 of the central core 16. The proximal face of the distal part 56 of the annular locking device 50 presses on the distal edge of the distal tube 14. The distal face of the pilot point 48 fitted into the proximal face 18 of the central core 16 presses on the distal face 13 of the transverse partition of the distal terminal end 11.

During a next locking phase, the removable head 31 in the distal terminal end 11 is rotated 45° anticlockwise. At the end of the locking phase, the respective positions of the various elements of the distal terminal end and of the removable head are in the following configuration.

The two radial fingers 51 fitted into the proximal part 53 of the annular locking device 50 press on the ends of the transverse arms 44 of the bayonet-shaped slits 42, 44 made in the distal tube 14. The point 48 fitted into the proximal face 18 of the central core 16 is housed in the blind cylindrical orifice 45 made in the transverse partition 13 of the distal terminal end 11. The proximal face 18 of the central core 16 presses on the transverse partition 13 of the distal terminal end 11. The contact pads 27 fitted into the proximal face 18 of the central core 16 come into contact with the contact pads 28 fitted into the transverse partition 13 of the distal terminal end 11. The coil spring 55 housed in the distal part 56 of the annular locking device 50 is slightly slack compared to the previous compression phase, such that the radial finger 49 fitted into the distal part 47 of the central core 16 is positioned in the median part of the slit 52 made in the distal part 56 of the annular locking device 50.

The locking thus obtained removes any possibility of accidental unlocking. The unlocking of the removable head 31 indeed requires the user to push the finger 49 away, using a sharp tool, towards the distal end of the slit 52 before turning the removable head 31 by 45° clockwise, so as to release the fingers 51 from the bayonet-shaped slits 42, 44 made in the distal end of the tube 14.

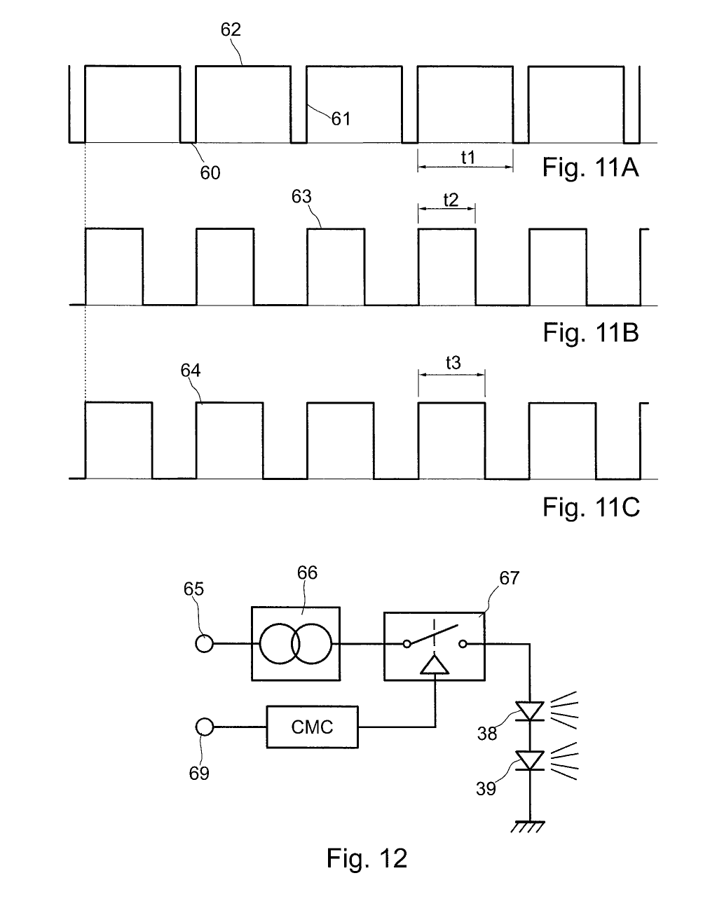

FIGS. 11A to 11C represent timing diagrams of control signals applied to the image sensor of the videoendoscopic probe when the sensor is of “interline transfer tricolor” CCD type. FIG. 11A represents line synchronizing pulses 62, and frame synchronizing pulses 60 emitted with a period equal to t1, i.e. 20 ms in PAL standard. FIG. 11B represents integrating pulses 63 of a duration equal to t2 and synchronous with the pulses 60.

The potential troughs of the light-sensitive layer of the image sensor 1 are only loaded during activation periods of duration t2 of the integrating signal 63. Therefore, the efficiency of the lighting device in terms of lighting is not substantially altered if the lighting device supplies, not a continuous light but a pulsed light of the same intensity comprising light pulses synchronous with the pulses 63 and having a duration equal to or greater than the activation duration t2 of the integrating signal.

Therefore, FIG. 11C represents the control pulses 64 for controlling the conduction of the LEDs 38, 39. The pulses 64 have a duration t3 equal to or slightly greater than the duration t2 and are also synchronous with the pulses 60.

With identical current intensities, the diodes 38, 39 powered in pulsed mode supply lighting substantially identical to that supplied in continuous mode, but with a lesser temperature rise. With identical current power and thus temperature rise, the diodes 38, 39 supply lighting in pulsed mode greater than that supplied in continuous mode.

FIG. 12 represents a schematic diagram of the pulsed supply device for powering the LEDs 38, 39. The supply device comprises a current generator 66 supplying a current of constant intensity which powers the diodes 38, 39 hard-wired in series through an electronic switch 67. An input 65 of the generator 66 is linked to a direct voltage source.

The control circuit of the switch 67 comprises a monostable circuit CMC supplying a pulse of duration t3 when a short pulse is applied to an input 69 of the monostable circuit. The short pulse is synchronous with the rising edge 61 of the frame synchronization signal 60.

It will be understood by those skilled in the art that various alternative embodiments and applications of the present invention may be made. In particular, the present application describes an axial-viewing removable head having a compact optical system, a pair of LEDs, and a locking device enabling the head to be associated with the distal terminal end of a videoendoscopic probe equipped with an image sensor of the “interline transfer tricolor” CCD type centered on the mechanical axis of the terminal end. It goes without saying that the present invention can also be applied to an endoscopic probe implementing another image capture technology than the one described above, or in which the optical axis of the image sensor integrated into the distal terminal end is not merged with the mechanical axis of the terminal end, or even to a fixed head endoscopic probe in which the optical system and the lighting device described in the present invention are integrated into the distal terminal end of the probe.

The present invention can also be applied to a deviated viewing removable head comprising a distal reflecting prism added to the optical system described previously, and the two LEDs being offset in the distal end of the removable head 31 so that the axes of the light beams of the LEDs are parallel to the viewing axis of the removable head.

The present invention can also be applied to the removable heads of endoscopic probes integrating an optical image splitting component, such as a delta prism for example, or an auxiliary image projecting device comprising for example a laser diode or an optical collimator intended to transmit the laser beam transmitted by a fiber integrated into the videoendoscopic probe.

In the case of endoscopic probes provided to be associated with a removable distal head, the objective is not necessarily entirely housed in the removable head. For example, the lens 5 of the objective can be housed in the distal terminal end of the probe.

Furthermore, it is not essential to provide two LEDs in the removable head. A single LED is sufficient if the LED supplies a light intensity sufficient for the intended use of the endoscopic probe.

In addition, if the lighting device chosen so permits, it is not necessary to reduce the section of the median lens 6 to house the lighting device. Using a distal lens of reduced section can be sufficient in certain cases. On the other hand, it is important that the proximal lens has a section corresponding to the light-sensitive surface of the image sensor and that the distal lens has a reduced section compared to the section of the proximal lens to house a lighting device supplying sufficient candle power.

It is not essential either for the two lenses 32, 33 having a reduced section compared to the proximal lens 5 to have the same section. If the lighting device used so permits, the median lens can have a larger section than that of the distal lens.

All the lenses of the objective can be machined so as to have a section corresponding to that of the light beam passing through them and covering the entire light-sensitive surface of the image sensor. In practice, the lenses of the objective have two different sections, one section corresponding to the light-sensitive surface for the proximal lens(es) and one section corresponding to the largest section of the distal part of the light beam passing through the distal part of the objective for the distal lenses.

The present invention can also be applied to other types of objectives than the one described above. Therefore, the objective can have more lenses and/or an arrangement of converging and diverging lenses different from the one described above.

The present invention can also be applied to the integration of devices other than a lighting device into the distal end of an endoscopic probe. Thus, reducing the dimensions of the objective can enable for example a laser diode of a metrology device, and possibly a single lighting diode, to be housed.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

I claim:

1. An endoscopic probe comprising a distal end comprising an image sensor having a substantially rectangular light-sensitive surface associated with an objective arranged for transmitting a light beam having a proximal section substantially identical to a light-sensitive surface of the image sensor,
wherein the objective comprises at least one first distal lens produced from a circular lens, and wherein parts of the circular lens that are not passed through by light rays transmitted by the objective to the light-sensitive surface have been removed, so as to reduce lateral dimensions of a distal part of the objective.

2. The endoscopic probe according to claim 1, wherein the first distal lens of the objective is produced from a circular lens having undergone two symmetrical lateral milling operations, a distance between them being substantially equal to a width of the widest section of a part of the light beam passing through the distal part of the objective and covering the light-sensitive surface.

3. The endoscopic probe according to claim 1, wherein the objective comprises a second distal lens having a section identical to that of the first distal lens.

4. The endoscopic probe according to claim 1, wherein the objective comprises at least one proximal lens of circular section, whose diameter is substantially identical to a diagonal of the light-sensitive surface.

5. The endoscopic probe according to claim 1, wherein the objective comprises at least one proximal lens machined from a circular lens, so as to have a width substantially identical to that of the light-sensitive surface.

6. The endoscopic probe according to claim 1, wherein the objective comprises a first diverging distal lens, a second converging distal lens, and a converging proximal lens, the distal lenses being produced from circular lenses, each having undergone two symmetrical lateral milling operations, a distance between them being substantially equal to a width of a widest section of a distal part of the light beam passing through the objective and covering the light-sensitive surface.

7. The endoscopic probe according to claim 1, comprising a lighting device comprising one or two high-power light-emitting diodes housed at least partly in a volume of the parts removed from the distal lens.

8. The endoscopic probe according to claim 7, wherein the lighting device comprises for each light-emitting diode a field lens, a printed circuit supporting the diode and a distal port protecting the diode.

9. The endoscopic probe according to claim 1, wherein the image sensor is housed in a distal end of the endoscopic probe.

10. The endoscopic probe according to claim 7, wherein at least one distal part of the objective and the lighting device are disposed in a head arranged for being fixed in a removable manner onto a distal terminal end of the endoscopic probe.

11. The endoscopic probe according to claim 10, wherein the removable head comprises a first electrical connection arranged for cooperating with corresponding electrical connection provided in the distal terminal end to ensure electrical connection of the lighting device when the removable head is fixed onto the distal terminal end.

12. The endoscopic probe according to claim 7, comprising a source of power in pulsed current to deactivate the lighting device when the image sensor is not in an acquisition phase.

13. A removable head lockable onto an endoscopic probe distal terminal end, the head comprising at least one distal part of an objective arranged for transmitting a light beam covering a substantially rectangular light-sensitive surface of an image sensor,
wherein the objective comprises at least one first distal lens produced from a circular lens, and wherein parts of the circular lens that are not passed through by light rays transmitted by the objective to the light-sensitive surface have been removed, so as to reduce lateral dimensions of the distal part of the objective.

14. The head according to claim 13, wherein the first distal lens of the objective is produced from a circular lens having undergone two symmetrical lateral milling operations, a distance between them being substantially equal to a width of a widest section of a part of the light beam passing through the distal part of the objective and covering the light-sensitive surface.

15. The head according to claim 13, wherein the objective comprises a second distal lens having a section identical to that of the first distal lens.

16. The head according to claim 13, wherein the objective comprises at least one proximal lens of circular section, whose diameter is substantially identical to a diagonal of the light-sensitive surface.

17. The head according to claim 13, wherein the objective comprises at least one proximal lens machined from a circular lens, so as to have a width substantially identical to that of the light-sensitive surface.

18. The head according to claim 13, wherein the objective comprises a first diverging distal lens, a second converging distal lens, and a converging proximal lens, the distal lenses being produced from circular lenses each having undergone two symmetrical lateral milling operations, a distance between them being substantially equal to a width of a widest section of a distal part of the light beam passing through the objective and covering the light-sensitive surface.

19. The head according to claim 13, comprising a lighting device comprising one or two high-power light-emitting diodes housed at least partly in a volume of the parts removed from the distal lens.

20. The head according to claim 19, wherein the lighting device comprises for each light-emitting diode a field lens, a printed circuit supporting the diode and a distal port protecting the diode.

21. The head according to claim 19, comprising a first electrical connection arranged for cooperating with a corresponding electrical connection provided in a distal terminal end of the probe to ensure electrical connection of the lighting device when the removable head is fixed onto the distal terminal end.
